(19) United States
(12) Patent Application Publication
(10) Pub. No.: US 2025/0049325 A1
(43) Pub. Date: Feb. 13, 2025

(54) SIMULTANEOUS MEASUREMENT OF TISSUE PERFUSION AND TISSUE OXYGENATION

(71) Applicant: KONINKLIJKE PHILIPS N.V., EINDHOVEN (NL)

(72) Inventors: Gerhardus Wilhelmus LUCASSEN, EINDHOVEN (NL); Rieko VERHAGEN, VUGHT (NL); Nikolaos TASIOS, EINDHOVEN (NL); Nico Maris Adriaan DE WILD, EINDHOVEN (NL); Willem VERKRUIJSSE, VELDHOVEN (NL); Marco LAI, EINDHOVEN (NL)

(21) Appl. No.: 18/718,075

(22) PCT Filed: Dec. 2, 2022
(86) PCT No.: PCT/EP2022/084291

§ 371 Date: Jun. 10, 2024

Related U.S. Application Data
(60) Provisional application No. 63/290,314, filed on Dec. 16, 2021.

(51) Int. Cl. A61B 5/00; A61B 5/1455

(52) U.S. Cl. CPC A61B 5/0077; A61B 5/14552

(57) ABSTRACT

A system includes a controller, a light source and a camera. The controller includes a memory that stores instructions and a processor that executes the instructions. The light source emits emission light towards tissue at six or more predetermined central wavelengths. The camera detects reflected light reflected from the tissue based on the emission light. When executed by the processor, the instructions cause the system to measure tissue perfusion based on the reflected light, and measure tissue oxygenation based on the reflected light.

### BACKGROUND

Tissue perfusion refers to blood flow in a tissue volume over time. Imaging photoplethysmography (iPPG) is an optical, contactless and non-invasive method to measure tissue perfusion. iPPG is based on the difference in reflected or transmitted light signals due to dynamic blood volume variations (blood flow perfusion) in living tissue of the vasculature. In iPPG, a camera measures periodic changes in skin reflectance, caused by the cardiovascular pressure waves passing through the skin microvasculature. The iPPG signal is typically expressed as AC/DC, where AC expresses the difference between minimum and maximum skin reflectance and DC expresses the average reflectance of the signals.

iPPG may also be used to measure arterial pulse oxygenation (SpO2), similar to arterial pulse oxygenation (Sp02) measured with pulse oximetry. However, in some clinical applications, such as for peripheral vascular disease, along with tissue perfusion, physicians need to measure tissue oxygenation (StO2) rather than arterial pulse oxygenation (SpO2). iPPG is currently not used to measure tissue perfusion and tissue oxygenation (StO2) simultaneously.

iPPG is clinically important in diagnosis, treatment and longitudinal monitoring of patients with peripheral vascular disease. Additionally, arterial pulse oxygenation (SpO2) measurements and tissue oxygenation (StO2) measurements may be performed before, during and/or after an intervention. However, although arterial pulse oxygenation (SpO2) is clinically very important, some clinicians suggest arterial pulse oxygenation (SpO2) measurements can be misleading in some cases of hemoglobin deviations such as in the case of high levels of methemoglobin or carboxyhemoglobin and in cases of low perfusion or when perfusion is obscured by severe edema or in case of shock.

Moreover, arterial pulse oxygenation (SpO2) expresses the condition of the cardiopulmonary system. Arterial pulse oxygenation (SpO2) informs how much O2 is provided to an organ; not how much O2 is taken up by the organ. In contrast, tissue oxygenation (StO2) is the oxygen saturation at the organ level, or capillary level, where the oxygen exchange takes place, and the capillary vessels do not feature pulsatility-causing PPG signals. As a result, measurement of tissue oxygenation (StO2) is typically performed with static (DC) techniques rather than PPG.

iPPG systems can be combined with hyperspectral imaging systems to perform imaging measurements at multiple wavelengths. For example, tissue oxygenation (StO2) may be calculated using diffuse reflectance spectroscopy measuring reflected light at 100 or more central wavelengths in the 700-1000 nm wavelength range, though this involves an extraordinary amount of processing for each instance of the tissue oxygenation (StO2) measurements. However, iPPG is currently not used to measure tissue perfusion and tissue oxygenation (StO2) simultaneously, such as using an optimized and minimized set of the same central wavelengths.

Another difficulty in the estimation of tissue oxygenation (StO2) from diffuse reflectance spectroscopy is that the differential path length factor (DPF), which is caused by the different paths of light of different wavelengths traveling through tissue, requires correction. For example green light and red light travel different paths, and estimation of tissue oxygenation (StO2) from these wavelengths would be incorrect as the light sees different parts of tissue. The DPF is currently not corrected even in the current pulse oximetry systems which use two wavelengths, and this can result in erroneous readings of the arterial pulse oxygenation (SpO2).

As set forth above, current PPG systems do not perform simultaneous tissue perfusion and tissue oxygenation (StO2) measurements. Clinically there is a need for a non-invasive and contactless mechanism for measurement of both tissue perfusion and tissue oxygenation (StO2).

### SUMMARY

According to an aspect of the present disclosure, a system includes a controller, a light source and a camera. The controller includes a memory that stores instructions and a processor that executes the instructions. The light source emits emission light towards tissue at 6 or more predetermined central wavelengths. The camera detects reflected light reflected from the tissue based on the emission light. When executed by the processor, the instructions cause the system to measure tissue perfusion based on the reflected light; and measure tissue oxygenation (StO2) based on the reflected light.

According to an aspect of the present disclosure, a device includes a controller, a light source and a camera. The controller includes a memory that stores instructions and a processor that executes the instructions. The light source emits emission light towards tissue at 6 or more predetermined central wavelengths. The camera detects reflected light reflected from the tissue based on the emission light. When executed by the processor, the instructions cause the system to measure tissue perfusion based on the reflected light; and measure tissue oxygenation (StO2) based on the reflected light.

According to an aspect of the present disclosure, a method of simultaneously measuring tissue perfusion and tissue oxygenation (StO2) includes storing instructions in a memory and executing the instructions by a processor. The method also includes emitting emission light from a light source towards tissue at 6 or more predetermined central wavelengths, and detecting reflected light reflected from the tissue based on the emission light at a camera. When executed by the processor, the method includes the system measuring tissue perfusion based on the reflected light; and measuring tissue oxygenation (StO2) based on the reflected light.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates a system for simultaneous measurement of tissue perfusion and tissue oxygenation, in accordance with a representative embodiment.

FIG. 1B illustrates a controller for simultaneous measurement of tissue perfusion and tissue oxygenation, in accordance with a representative embodiment.

FIG. 2A illustrates a method for simultaneous measurement of tissue perfusion and tissue oxygenation, in accordance with a representative embodiment.

FIG. 2B illustrates a method for simultaneous measurement of tissue perfusion and tissue oxygenation, in accordance with a representative embodiment.

FIG. 3A illustrates a comparison of calculated tissue oxygenation (StO2) versus ground truth tissue oxygenation (StO2) with DPF correction and without DPF correction.

FIG. 3B illustrates a dual-layer diffuse scattering skin model for simultaneous measurement of tissue perfusion and tissue oxygenation, in accordance with a representative embodiment.

FIG. 4 illustrates building a set of DRS spectra as input to a CNN model for simultaneous measurement of tissue perfusion and tissue oxygenation, in accordance with a representative embodiment.

FIG. 5 illustrates a total set of diffuse spectra used for a CNN model for simultaneous measurement of tissue perfusion and tissue oxygenation, in accordance with a representative embodiment.

FIG. 6 illustrates a Monte-Carlo model used to generate a set of DRS spectra as input to a CNN model for simultaneous measurement of tissue perfusion and tissue oxygenation, in accordance with a representative embodiment.

FIG. 7 illustrates a DeepSkin CNN model used for skin parameter extraction for simultaneous measurement of tissue perfusion and tissue oxygenation, in accordance with a representative embodiment.

FIG. 8 illustrates a DeepSkin CNN model applied to hyperspectral data from facial skin images to extract physiological parameters of skin for simultaneous measurement of tissue perfusion and tissue oxygenation, in accordance with a representative embodiment.

FIG. 9 illustrates a 1-dimensional CNN for testing performance of parameter estimation from DRS spectra for simultaneous measurement of tissue perfusion and tissue oxygenation, in accordance with a representative embodiment.

FIG. 10 illustrates predicted tissue oxygenation and predicted total hemoglobin for simultaneous measurement of tissue perfusion and tissue oxygenation, in accordance with a representative embodiment.

FIG. 11 illustrates optimal wavelength sample point sets for StO2, HbT and MetHb for simultaneous measurement of tissue perfusion and tissue oxygenation, in accordance with a representative embodiment.

FIG. 12 illustrates CNN model performance for simultaneous measurement of tissue perfusion and tissue oxygenation, in accordance with a representative embodiment.

FIG. 13 illustrates a system for simultaneous measurement of tissue perfusion and tissue oxygenation, in accordance with a representative embodiment.

FIG. 14 illustrates another system for simultaneous measurement of tissue perfusion and tissue oxygenation, in accordance with a representative embodiment.

FIG. 15 illustrates a computer system, on which a method for simultaneous measurement of tissue perfusion and tissue oxygenation is implemented, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described herein, a contactless and non-invasive system is provided for measuring tissue perfusion and tissue oxygenation (StO2) simultaneously. One implementation uses an optimized, selected and minimal set of wavelengths to ensure accurate estimations of the tissue perfusion and tissue oxygenation (StO2). One advantage of simultaneous measuring of both tissue perfusion and tissue oxygenation (StO2) with a single system is that, even in cases of low perfusion or low pulsatility, the DC signal (spectrum) still reflects tissue oxygenation (StO2). Otherwise, in cases of low perfusion or low pulsatility, iPPG may not be able to discern a small PPG signal from noise. The accurate measurement of tissue oxygenation (StO2) using reflectance mode PPG requires measurement of the diffuse reflectance at multiple wavelengths. The teachings herein provide an optimized, selected and minimal set of wavelengths that ensure a certain accuracy in determining the tissue oxygenation (StO2) from the PPG imaging spectra.

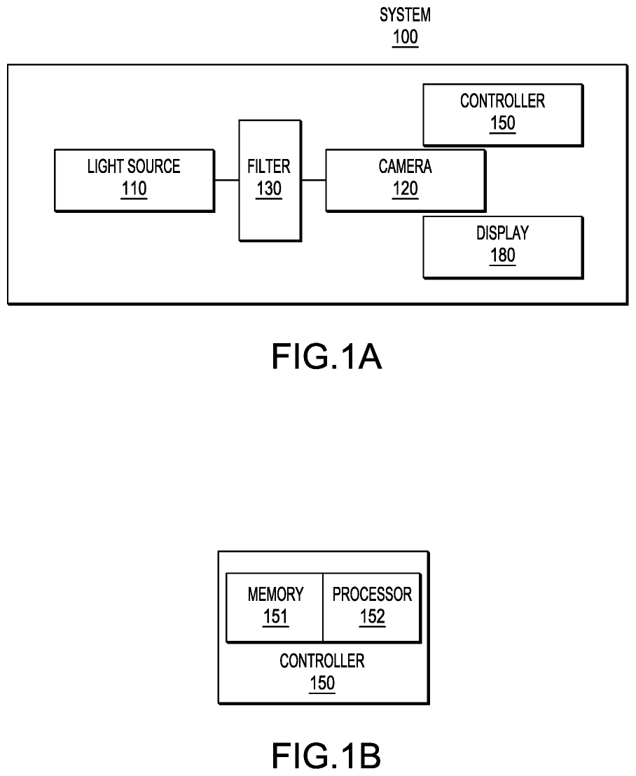

FIG. 1A illustrates a system 100 for simultaneous measurement of tissue perfusion and tissue oxygenation, in accordance with a representative embodiment.

The system 100 in FIG. 1A is a system for simultaneous measurement of tissue perfusion and tissue oxygenation and includes components that may be provided together or that may be distributed. The system 100 includes a light source 110, a camera 120, a filter 130, a controller 150, and a display 180. Although the components of the system 100 shown in FIG. 1A appear to be coplanar, they are not necessarily so. Nor are the components of the system 100 necessarily physically attached or separated in the manner shown in FIG. 1A. The system 100 is, however, configured to be contactless and operate without contact relative to a subject of the imaging performed by the system 100.

The light source 110 may be a broadband light source, in which case the light source emits broadband emission light that includes light at the 6 or more predetermined central wavelengths. The broadband emission light may be white light. The light source 110 may alternatively be a multi-wavelength light source specifically configured to emit light centered at 6 or more predetermined individual central wavelengths, or a hyper-wavelength light source specifically configured to emit light centered at a larger number of predetermined individual central wavelengths than a multi-wavelength light source. The emission light emitted at central wavelengths may have bandwidths around the central wavelengths within a few nm, e.g., 2-3 nm, and a full width at half maximum of, e.g., 10-20 nm. Alternatively, the light source 110 may comprise lasers that emit emission light at the predetermined central wavelengths at more focused and tighter bandwidths.

The camera 120 may be configured to perform imaging photoplethysmography (iPPG). The camera 120 may be provided with the filter 130, or the camera 120 and the filter 130 may be provided as separate, or at least separable, components of the system 100.

The camera 120 in FIG. 1A may be provided without the filter 130, such as when the camera 120 is a multispectral camera configured to detect the 6 or more predetermined central wavelengths as a fixed configuration. The 6 or more central wavelengths are optimized in a manner extensively described below. Alternatively, the camera 120 may be provided with the filter 130, to limit the reflected light received by the camera 120 to reflected light at 6 or more central wavelengths as described herein. In some embodiments, the camera 120 may be configured to detect reflected light at 6 or more central wavelengths in one mode, and may be configured to detect broadband light reflected in another mode.

In some embodiments, the camera 120 is a hyperspectral camera configured to detect the 6 or more central wavelengths as a tunable configuration. In other embodiments, the camera 120 is a hyperspectral camera and the filter 130 is configured to filter the reflected light to the 6 or more central wavelengths as a tunable configuration.

The filter 130 may be provided to selectively pass light at 6 or more central wavelengths at the same time, or may be a wheel filter configured to selectively pass light at 1 of 6 or more central wavelengths at a time. For example, the filter 130 may be configured to simultaneously filter the reflected light to 6 predetermined central wavelengths.

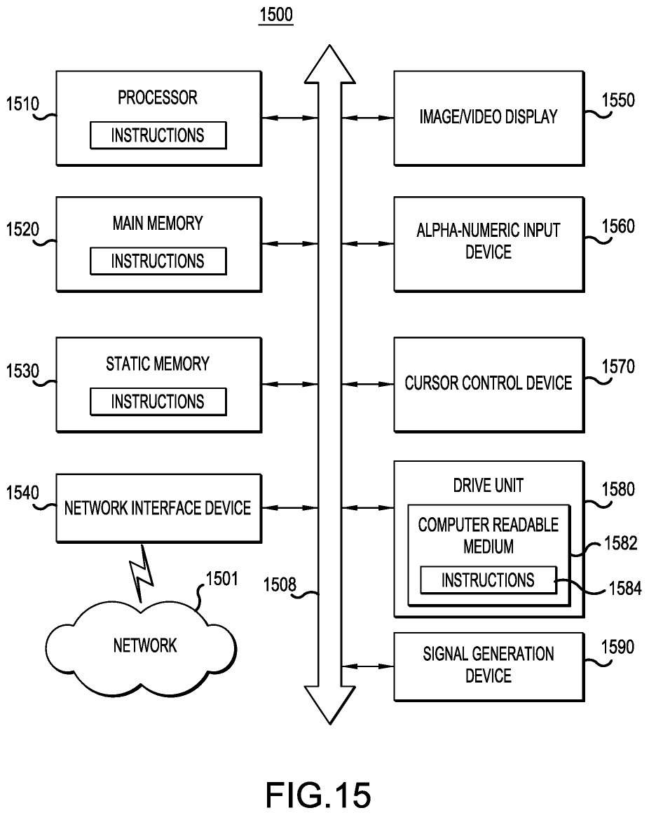

The controller 150 is further depicted in FIG. 1B, and includes at least a memory 151 that stores instructions and a processor 152 that executes the instructions. A computer that can be used to implement the controller 150 is depicted in FIG. 15, though a controller 150 may include more or fewer elements than depicted in FIG. 1B or in FIG. 15.

The display 180 may be local to the controller 150 or may be remotely connected to the controller 150. The display 180 may be connected to the controller 150 via a local wired interface such as an Ethernet cable or via a local wireless interface such as a Wi-Fi connection. The display 180 may be interfaced with other user input devices by which users can input instructions, including mouses, keyboards, thumbwheels and so on.

The display 180 may be a monitor such as a computer monitor, a display on a mobile device, an augmented reality display, a television, an electronic whiteboard, or another screen configured to display electronic imagery. The display 180 may also include one or more input interface(s), as well as an interactive touch screen configured to display prompts to users and collect touch input from users.

FIG. 1B illustrates a controller 150 for simultaneous measurement of tissue perfusion and tissue oxygenation, in accordance with a representative embodiment.

The controller 150 includes a memory 151 and a processor 152. The memory 151 stores instructions which are executed by the processor 152. The processor 152 executes the instructions.

The controller 150 may perform some of the operations described herein directly and may implement other operations described herein indirectly. For example, the controller 150 may indirectly control other operations such as by generating and transmitting content to be displayed on the display 180. Accordingly, the processes implemented by the controller 150 when the processor 152 executes instructions from the memory 151 may include steps not directly performed by the controller 150.

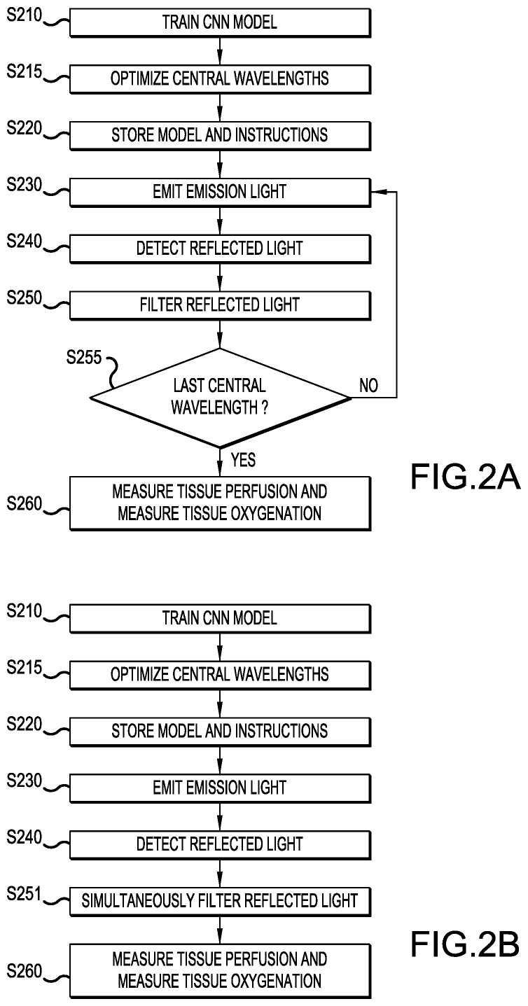

FIG. 2A illustrates a method for simultaneous measurement of tissue perfusion and tissue oxygenation, in accordance with a representative embodiment.

The method of FIG. 2A may be performed by the controller 150, or by the system 100 including the controller 150.

The method of FIG. 2A starts with training a convolutional neural network (CNN) model at S210.

At S215, central wavelengths are optimized. For the purposes of the teachings herein, 6 or more central wavelengths are identified and selected as an optimal set. Minimizing the number of optimized central wavelengths is useful in terms of minimizing the processing and storage requirements for reflected light detected by cameras, so there is some benefit to maintaining the number of optimized central wavelengths to exactly 6 and, if not, to 7, and so on. The 6 or more predetermined central wavelengths are optimized for simultaneous detection of tissue perfusion, tissue oxygenation, total hemoglobin and methemoglobin.

At S220, the CNN model and instructions for the processor are stored.

At S230, emission light is emitted. The emission light may be white light, may be multispectral light, may be hyper-spectral light, or may be emission light emitted only at the 6 or more central wavelengths described herein.

At S240, reflected light is detected.

At S250, reflected light is filtered. Although the filtering is shown after the detection in the method of FIG. 2A, the filtering may be performed before the detection in some embodiments described herein.

At S255, a determination is made as to whether the detected light which is filtered is the last central wavelength to be detected.

If the detected light which is filtered is the last central wavelength (S255=Yes), at S260 the method of FIG. 2A includes measuring tissue perfusion and measuring tissue oxygenation based on the reflected light detected at the 6 or more optimized central wavelengths. The tissue perfusion and the tissue oxygenation may be measured based on the reflected light independent of interferences due to characteristics of the tissue.

At S260, the measuring may be performed by applying the trained convolutional neural network to the reflected light. The trained CNN may be applied to obtain an output comprising an estimate of the tissue perfusion, an estimate of tissue oxygenation, an estimate of total hemoglobin and an estimate of methemoglobin.

If the current central wavelength is not the last central wavelength (S255=No), the process returns to S230 and the process is performed until the last central wavelength is detected and filtered so that the tissue perfusion and tissue oxygenation may be measured at S260.

In the method of FIG. 2A, reflected light is detected at isolated central wavelengths, one at a time, such as when a filter wheel is used as the filter 130 in FIG. 1A.

FIG. 2B illustrates another method for simultaneous measurement of tissue perfusion and tissue oxygenation, in accordance with a representative embodiment.

The method of FIG. 2B may be performed by the controller 150, or by the system 100 including the controller 150.

The method of FIG. 2BA again starts with training a convolutional neural network (CNN) model at S210.

At S215, central wavelengths are optimized. The 6 or more predetermined central wavelengths are optimized for simultaneous detection of tissue perfusion, tissue oxygenation, total hemoglobin and methemoglobin.

At S220, the CNN model and instructions for the processor are stored.

At S230, emission light is emitted. The emission light may be white light, may be multispectral light, may be hyper-spectral light, or may be emission light emitted only at the 6 or more central wavelengths described herein.

At S240, reflected light is detected.

At S251, reflected light is simultaneously filtered. Although the filtering is shown after the detection in the method of FIG. 2B, the filtering may be performed before the detection in some embodiments described herein.

At S260 the method of FIG. 2B includes measuring tissue perfusion and measuring tissue oxygenation based on the reflected light detected at the 6 or more optimized central wavelengths. The tissue perfusion and the tissue oxygenation may be measured based on the reflected light independent of interferences due to characteristics of the tissue.

At S260, the measuring may be performed by applying the trained convolutional neural network to the reflected light. The trained CNN may be applied to obtain an output comprising an estimate of the tissue perfusion, an estimate of the tissue oxygenation, an estimate of total hemoglobin and an estimate of methemoglobin.

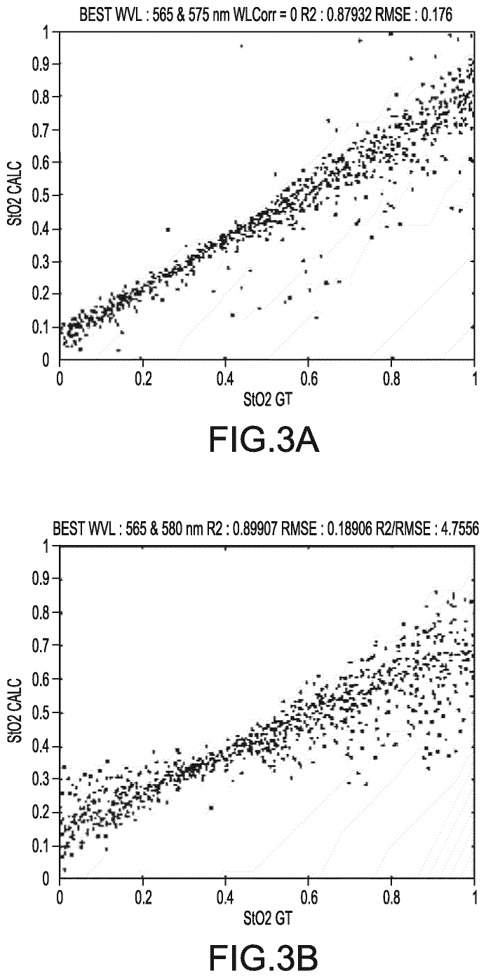

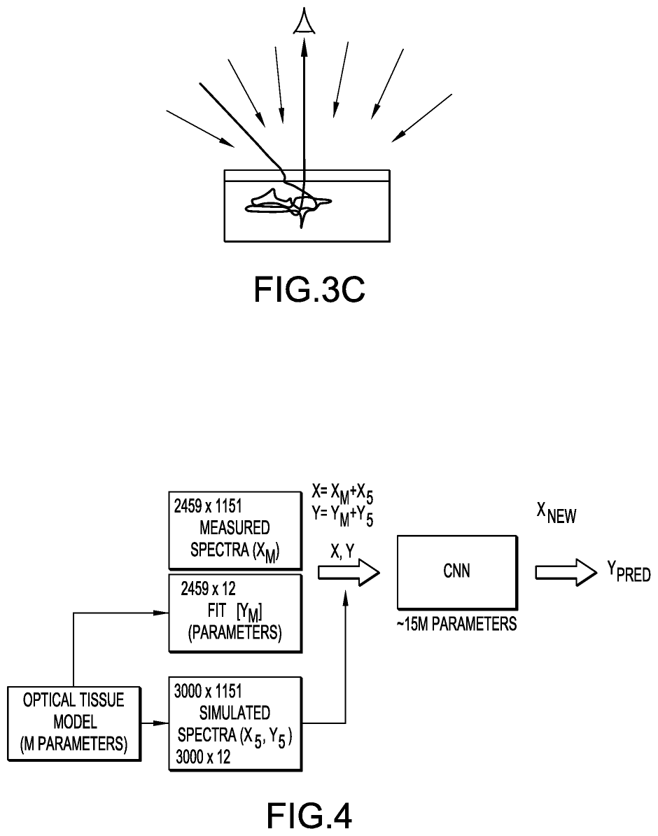

FIG. 3A illustrates a comparison of calculated tissue oxygenation (StO2) versus ground truth tissue oxygenation (StO2) with DPF correction and without DPF correction.

To address the issue of differential pathlength factor (DPF) at different wavelengths, the effect of DPF correction to the estimation of tissue oxygenation (StO2) may be investigated from simulated diffuse reflectance spectra (DRS) using a modified Farrell tissue model. Estimation of tissue oxygenation (StO2) from the absorption coefficients of Hb and HbO2 is given by Equation 1:

$$St O 2 = \frac{{e}_{HB}^{1} - {e}_{HB}^{2} {ρ}_{1} / {ρ}_{2}}{( {e}_{HB}^{1} - {e}_{HBO}^{1} ) - ( {e}_{HB}^{2} - {e}_{HBO}^{2} ) {ρ}_{1} / {ρ}_{2}} \qquad Eq 1$$

$${ρ}_{1} / {ρ}_{2} = \frac{A 1 DPF 2}{A 2 DPF 1}$$

$$DPF ( λ ) = \frac{1}{2} {( \frac{3 {μ}_{s}^{′} ( λ )}{{μ}_{a} ( λ )} )}^{1 / 2} [ 1 - \frac{1}{( 1 + {d ( 3 {μ}_{a} ( λ ) {μ}_{s}^{′} ( λ ) )}^{1 / 2} )} ]$$

In Equation 1, the eHB and eHBO are the molar extinction coefficients of Hb and HbO2 respectively, A1 and A2 are the log of the Reflectance at wavelengths 1 and 2, DPF1 and DPF2 are the DPFs at these wavelengths 1 and 2.

A modified Farrell DRS model may be used to generate N=3000 reflectance spectra where various tissue model parameters may be varied randomly within physiological ranges, such as total hemoglobin HbT, tissue oxygenation (StO2), water concentration, fat concentration, beta-carotene concentration, methemoglobin concentration and collagen concentration, and scattering amplitude, Mie and Rayleigh scattering factors. A database of absorption spectra of the different chromophores over the 400-1700 nm wavelength range and known wavelength dependencies of scattering may be input to the model. In addition 1% noise may be added to the simulated spectra.

To find the set of wavelengths 1 and 2 that give the best calculated tissue oxygenation (StO2) compared to the ground truth (input) tissue oxygenation (StO2) a correlation map (see the left side of FIG. 3A) may be generated based on running across all wavelengths in the 400-1700 nm range based on correlation coefficient R2=corrcoef(StO2GT,StO2) and the root mean squared error RMSE=sqrt(mean((StO2-StO2GT).\hat{ }2)) of the calculated tissue oxygenation (StO2) from the equation 1 given above against the tissue oxygenation (StO2) ground truth simulation input values. The set which gives the highest R2 may be selected and the tissue oxygenation (StO2) calculated versus tissue oxygenation (StO2) GT plot generated (see the right side of FIG. 3A).

FIG. 3A shows calculated tissue oxygenation (StO2) versus ground truth tissue oxygenation (StO2) with DPF correction (left) and without DPF correction (right). DPF correction gives a slight improvement on the R2/RMSE=5 versus 4.76.

From FIG. 3A it can be seen that DPF correction gives a slight improvement on the accuracy, where the metric correlation coefficient divided by the root mean squared error R2/RMSE value is used. With DPF correction this value is 5 and without 4.7.

Although the correlation coefficient is high, one can see from FIG. 3A that there is a large spread in calculated versus ground truth tissue oxygenation (StO2) in particular at relevant range 0.6-1.0.

FIG. 3B illustrates a dual-layer diffuse scattering skin model for simultaneous measurement of tissue perfusion and tissue oxygenation, in accordance with a representative embodiment.

In order to determine which wavelengths are optimal for estimation of tissue oxygenation, simulations of diffuse reflectance spectra may be obtained twofold, firstly by using a modified Farrell tissue model and secondly by using Monte Carlo simulations. The Monte Carlo simulations may use a dual layer diffuse scattering skin model. In the dual-layer diffuse scattering skin model of FIG. 3, uniform illumination results in a combination of Mie and Rayleigh scattering in dermis. The model parameters include incident illumination, melanin concentration, beta-carotene concentration, oxyhemoglobin, deoxy hemoglobin and methemoglobin concentrations, water, and lipid concentrations.

FIG. 4 illustrates building a set of DRS spectra as input to a CNN model for simultaneous measurement of tissue perfusion and tissue oxygenation, in accordance with a representative embodiment.

Straightforward matching with a model may involve minimization of a residue between measured and modelled DC reflectance spectra, and may lead to large uncertainties and errors in the tissue parameters. Therefore, more advanced methods are necessary such as machine learning. Simulations may be input to a convolutional neural network (CNN) and split into a training, validation and independent test set to test performance of estimating the ground truth parameters including tissue oxygenation tissue oxygenation (StO2) in the presence of other interferences (all the other tissue parameters). In FIG. 4, the set of DRS spectra are built as input to the CNN model. The simulated DRS spectra Xs may be obtained by generating 3000 spectra with 1151 wavelengths (450-1600 nm) using the modified Farrell optical tissue model varying 12 model (tissue) parameters ys. A set of measured spectra Xm N=2459 may be added to the simulated set. The fit parameters ym to the measured spectra and the known simulation parameters ys combined are the ground truth y for the input into the CNN model. In the CNN model the data set is split into a Training, Validation, and testing set. The CNN model may consist of a 1-dimensional convolution neural net layer with 15 M features (parameters). After training and validating the CNN model yields the prediction of the tissue parameters ypred on new input spectra Xnew as the test set of spectra.

Estimating the parameters may be initially performed on the whole spectral available range. By applying a wavelength selection algorithm, the CNN model performance may be tested on limiting the wavelength range to the selected wavelengths. Finally, performance as a function of number of selected wavelengths may yield the optimum minimal set of wavelengths to estimate tissue oxygenation (StO2) at a given accuracy.

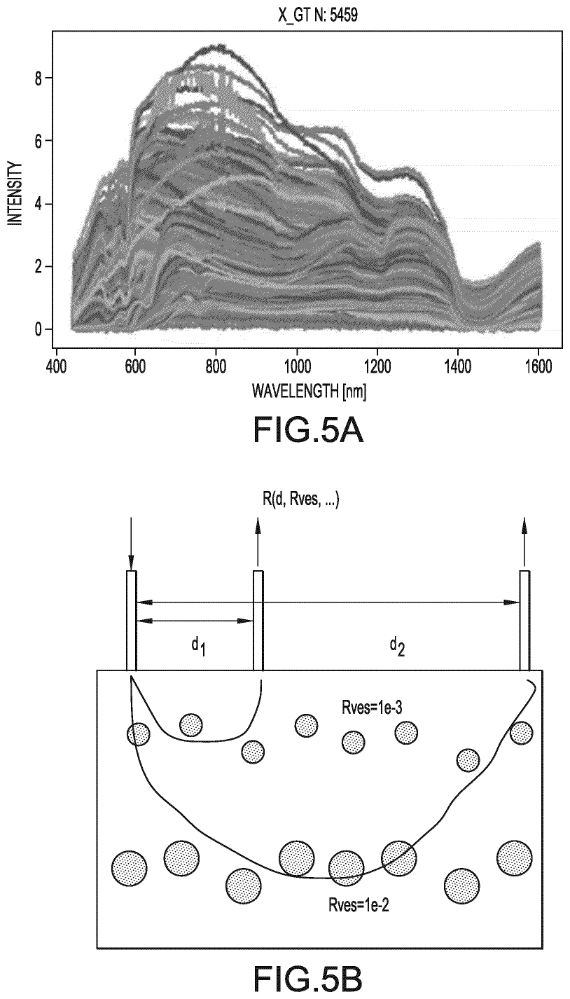

FIG. 5 illustrates a total set of diffuse spectra used for a CNN model for simultaneous measurement of tissue perfusion and tissue oxygenation, in accordance with a representative embodiment.

A modified Farrell model applicable to fiber probe DRS may be used to generate N=3000 spectra where various tissue model parameters are varied randomly within physiological ranges, such as total hemoglobin HbT, tissue saturation (StO2), water concentration, fat concentration, beta-carotene concentration, methemoglobin concentration and collagen concentration, and scattering amplitude, Mie and Rayleigh scattering factors. A database of absorption spectra of the different chromophores over the 400-1700 nm wavelength range and known wavelength dependencies of scattering may be input to the model. In addition, noise may be added to the simulated spectra. The set of simulated spectra may be complemented with measured tissue reflectance spectra N=2459. The measured spectra may be fitted to obtain ground truth parameters.

In FIG. 5, a total set of diffuse spectra consisting of simulated and measured tissue reflectance spectra used for the CNN mode is shown. Light source and geometry are included in the spectra in FIG. 5.

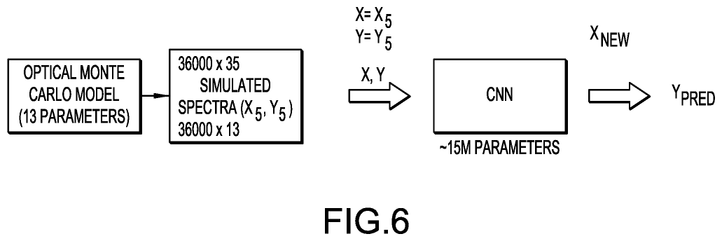

FIG. 6 illustrates a Monte-Carlo model used to generate a set of DRS spectra as input to a CNN model for simultaneous measurement of tissue perfusion and tissue oxygenation, in accordance with a representative embodiment.

In FIG. 6, a Monte-Carlo model is used to generate a set of DRS spectra as input to the CNN model. The simulated DRS spectra Xs may be obtained by generating 36000 spectra with 35 wavelengths (400-1100 nm) using an optical tissue model varying 13 model (tissue) parameters ys. The known input parameters ys are the ground truth y for the input into the CNN model.

In an experiment, diffuse reflectance data may be generated using a Monte-Carlo simulation applicable for imaging purposes. Assuming a geometry as is typical for a camera application whereby diffuse white light is incident on skin from all directions and the detection system is observing light emitted from the skin from a small area (one image pixel) under a single angle or small angular range (object space numerical aperture), according to the reciprocity theorem, the source and the detector may be swapped, resulting in an incident pencil beam of light on the skin combined with a hemispherical diffuse detector that collects the remitted photons for simulation purposes. The experiment may involve simulating a multilayer skin model incorporating 15 relevant parameters for the skin chromophores in the different layers and varying the chromophore concentrations over physiologically relevant values. In this experiment, over 36000 spectra may be generated using 35 different wavelengths as shown in FIG. 6.

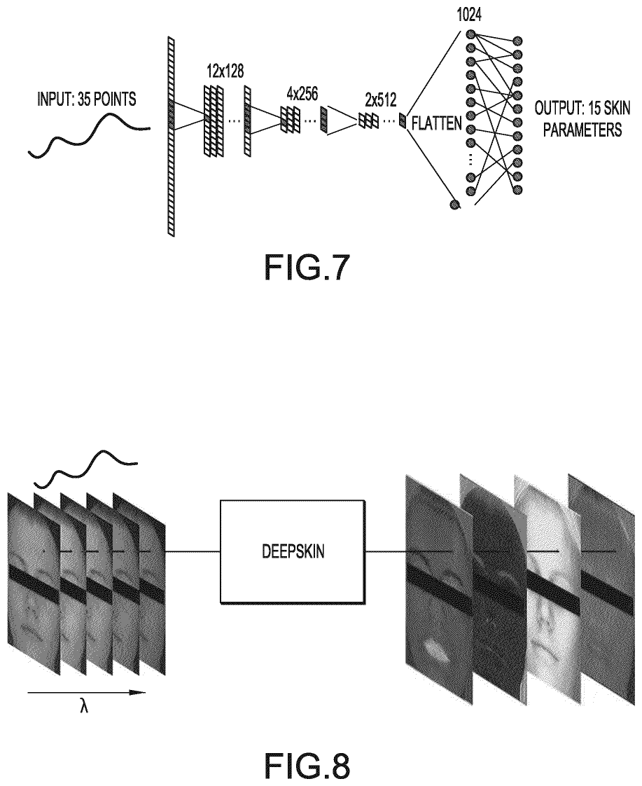

FIG. 7 illustrates a DeepSkin CNN model used for skin parameter extraction for simultaneous measurement of tissue perfusion and tissue oxygenation, in accordance with a representative embodiment.

FIG. 7 is a schematic representation of the DeepSkin CNN model used for skin parameter extraction. The accuracy of the model may be verified on the test set data for both numbers of wavelengths, and finds no significant difference between outcomes. The trained model may then be used to extract parameters from data obtained with a hyperspectral camera applied on a volunteer's facial skin. The data may be split in a training and test set and a CNN model referenced as “DeepSkin” as schematically depicted in FIG. 7. The CNN model may be s trained to extract the parameters. Various perturbations may then be applied to the data in order to improve the network stability and performance.

FIG. 8 illustrates a DeepSkin CNN model applied to hyperspectral data from facial skin images to extract physiological parameters of skin for simultaneous measurement of tissue perfusion and tissue oxygenation, in accordance with a representative embodiment.

In FIG. 8, a DeepSkin model is applied on hyperspectral data from facial skin images of volunteers to extract relevant physiological parameters of skin. The extracted chromophores may then be used to re-generate the spectra using the Monte Carlo simulation and these spectra may then be compared to the camera measured spectra for verification.

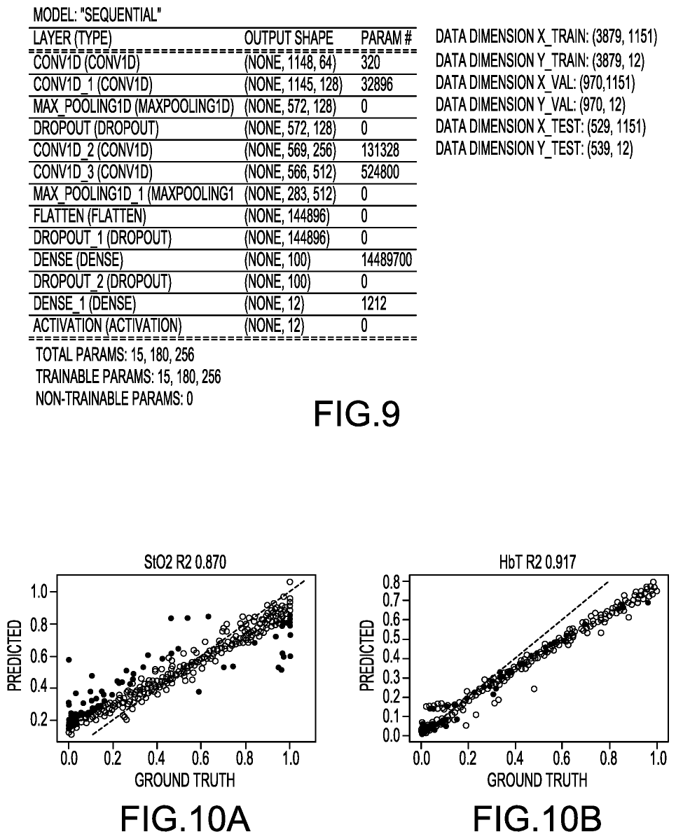

FIG. 9 illustrates a 1-dimensional CNN for testing performance of parameter estimation from DRS spectra for simultaneous measurement of tissue perfusion and tissue oxygenation, in accordance with a representative embodiment.

In FIG. 9, a simple 1-dimensional convolutional neural network is developed to estimate tissue parameters from the sets of generated DRS spectra. FIG. 9 shows the layer structure, including 2 convolution layers, 1 maxpooling layer, 2 convolution layers and 1 flatten layer to provide the dense layer output of 12 parameters. Dropout layers are used to address outliers. In total about 15 Million features are used in the model. The training, validation and test set split dimensions are also shown in FIG. 9.

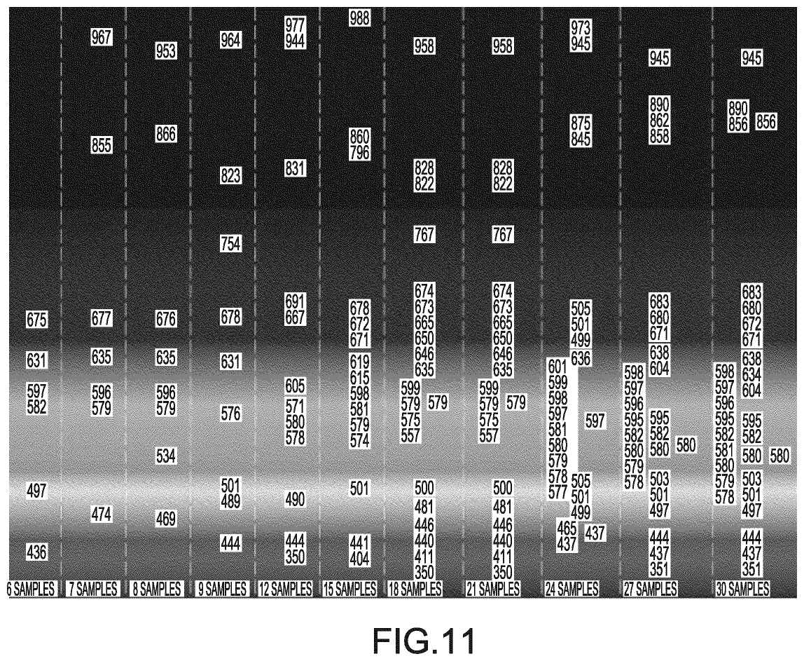

FIG. 10 illustrates predicted tissue oxygenation and predicted total hemoglobin for simultaneous measurement of tissue perfusion and tissue oxygenation, in accordance with a representative embodiment.

In FIG. 10, the predicted tissue oxygenation tissue oxygenation (StO2) and predicted total Hemoglobin HbT versus ground truths from the CNN model using the whole spectral range of 1151 wavelengths from 450-1600 nm are shown. The Piersson's correlation coefficient R2=0.87 for tissue oxygenation (StO2) prediction and R2=0.91 for HbT.

FIG. 11 illustrates optimal wavelength sample point sets for tissue oxygenation (StO2), HbT and MetHb for simultaneous measurement of tissue perfusion and tissue oxygenation, in accordance with a representative embodiment.

Several existing wavelength selection algorithms may be applied. For the teachings herein, an optimal sampling theory (Fisher analysis) is applied to perform wavelength selection to limit the number of wavelengths tuned to the optimal estimation of tissue oxygenation (StO2), total hemoglobin HbT and methemoglobin MetHb albeit in the presence of all other interferences (the other tissue parameters). A dual layer diffuse scattering skin model is used, with uniform diffuse illumination, no scattering in epidermis, combination of Mie and Rayleigh scattering in dermis. Model parameters are the incident illumination, melanin concentration, beta carotene concentration, concentrations of oxyhemoglobin, deoxyhemoglobin and methemoglobin, bilirubin concentration, water concentration and lipid (fat) concentration. The target is tissue oxygenation (StO2) optimization

$$α = \frac{{c}_{HbO 2}}{{c}_{HbO 2} + {c}_{Hb} + {c}_{MHb}}$$

This analysis uses a dual skin layer reflectance model:

$$I = {I}_{o} {T}_{epi} \sqrt{\frac{{a}^{‵} ( 1 + Q )}{2 P}} , in which P = ( 1 + \sqrt{3} R ) , Q = {e}^{- \frac{4 ζ R}{\sqrt{3}}} , R = \sqrt{1 - {a}^{‵} ,} {a}^{‵} = \frac{μ {′}_{s}}{μ {′}_{s} + {μ}_{a}} , ζ = 2.8 , {T}_{epi} = {e}^{- 2 {d}_{epi} \sum {ce}_{i} {μ}_{{a}_{i} epi}} ,$$

and μ_{α}=cdᵢμₐ_{ᵢ}ₐₑᵣ, whereby Tₑₚᵢ takes the filtering effect of the epidermis into account. The factors Tₑₚᵢ and Pa are wavelength dependent and are described by a sum of their relevant chromophore spectra, weighted by the relevant concentration term. Relevant independent parameters therefore are: i_{O}, dₑₚᵢceᵢ, cd₁. The results of the optimal sampling for different number of (whole valued) spectral points are shown in FIG. 11. In FIG. 11, optimal wavelength sample point sets for tissue oxygenation (StO2), HbT and MetHb estimation for different number of wavelengths are indicated in the top row.

In practice, the emission from the light source 110 at each of the optimized central wavelengths may have certain bandwidths around each respective central wavelength. The effects of bandwidth on the performance of the CNN model may be accounted for in the training. For example, optimized central wavelengths may have bandwidths of 1-3 nm, or 3-5 nm, or 3-15 nm. The same bandwidths may also be present for and applicable to each respective central wavelength at the filter 130 and at the detector of the camera 120.

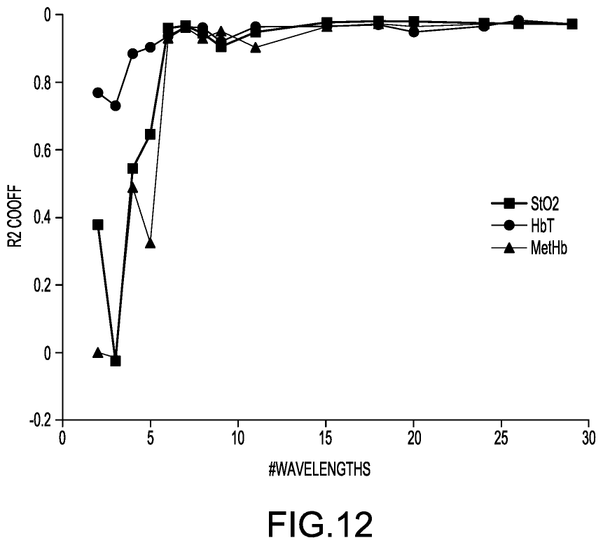

FIG. 12 illustrates CNN model performance for simultaneous measurement of tissue perfusion and tissue oxygenation, in accordance with a representative embodiment.

The results on the optimal wavelength sets may be tested using the CNN model. The (simulated) spectral input may be filtered on the wavelengths per set. The outcome of the CNN model may be predicted values for the tissue oxygenation (StO2), HbT and MetHb versus the given (ground truth) input parameters. The CNN model may be applied to the different sets of optimal wavelengths to see at what number of wavelengths the performance decreases. FIG. 12 shows that when the number of optimal wavelengths drops below 6, the performance (i.e., the R2 correlation coefficient of prediction versus ground truth) sharply decreases. This provides a practical boundary condition with respect to the performance of the parameter estimation. For R2 at least 0.8, say, simultaneous measurement of tissue perfusion and tissue oxygenation (StO2) effectively requires at least 6 wavelengths.

FIG. 12 shows CNN model performance (R2 correlation coefficient of predicted versus ground truth parameters) as a function of the number of wavelengths in the Fisher optimal wavelength sets. The performance i.e., the R2 coefficient for tissue oxygenation (StO2), HbT and MetHb decreases sharply when the number of wavelengths is less than 6.

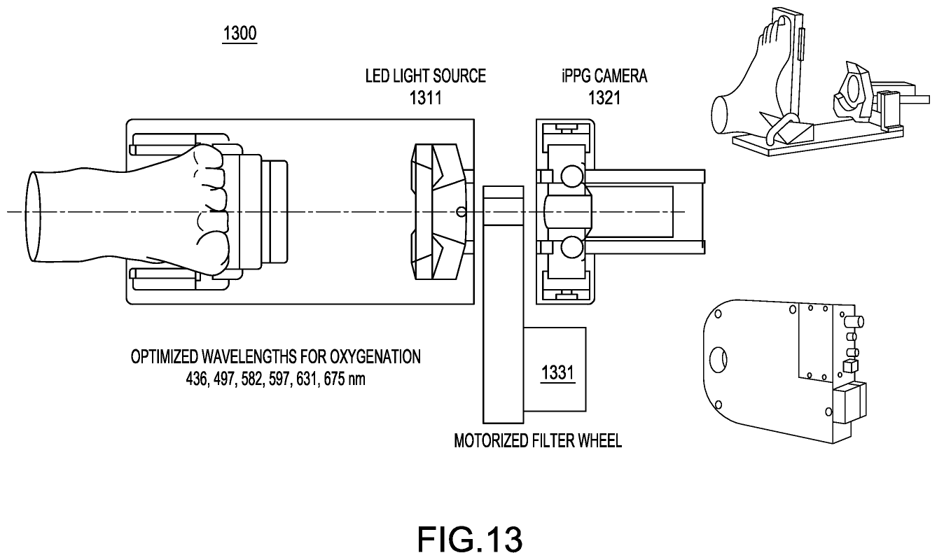

FIG. 13 illustrates a system for simultaneous measurement of tissue perfusion and tissue oxygenation, in accordance with a representative embodiment.

In embodiments based on FIG. 13, a system 1300 includes an LED light source 1311, a motorized filter wheel 1331, an iPPG camera 1321 and a personal computer (not labelled).

The iPPG camera 1321 performs imaging photoplethysmography on reflected light that passes through the motorized filter wheel 1331. The iPPG camera 1321 in FIG. 13 is used for measurements on a foot. The motorized filter wheel 1331 includes a set of wavelength filters and a filter wheel. The iPPG camera and the motorized filter wheel 1331 are used for simultaneously detecting blood pulsatility changes and tissue oxygenation (StO2) with multispectral measurements. With the motorized filter wheel 1331, the optimized central wavelengths may be passed to the iPPG camera 1321 one at a time or all at the same time.

The motorized filter wheel 1331 filters reflected light so that the iPPG camera 1321 only receives light at and around the optimized central wavelengths. The detected light that passes through the motorized filter wheel 1331 in FIG. 13 is multispectral light and is used for both measurements of blood volume pulsatility (PPG AC/DC signal over time) and for tissue oxygenation (StO2) estimation (DC component or the diffuse reflectance spectrum).

The LED light source 1311 is a light-emitting diode (LED) light source. The LED light source 1311 and the iPPG camera 1321 may be connected to the personal computer (not labelled) for measurement control, driving the motorized filter wheel 1331, for data acquisition and for data processing. The data processing includes applying a trained CNN model for tissue oxygenation (StO2), HbT and MetHb estimation. The central wavelengths of the motorized filter wheel 1331 are selected according to the optimized wavelength sets. With these selected wavelengths, the tissue oxygenation (StO2), total hemoglobin and methemoglobin parameters may be estimated with constant performance in the presence of interferences due to other parameters such as varying scattering, water and fat concentrations. The constant performance may be shown as an R2 correlation with ground truth parameters.

The LED lights source 1311 is a multi-wavelength light source. In practice, the wavelength filters of the motorized filter wheel 1331 may have bandwidths around central wavelengths within a few nm, e.g., 2-3 nm, and a full width at half maximum of, e.g., 10-20 nm. The impact of wavelength shifts caused by reflected light incident under angles different from perpendicular on the iPPG camera 1321 and filters of the motorized filter wheel 1331 may be accounted for in the training of the CNN model to estimate tissue oxygenation (StO2) etc.

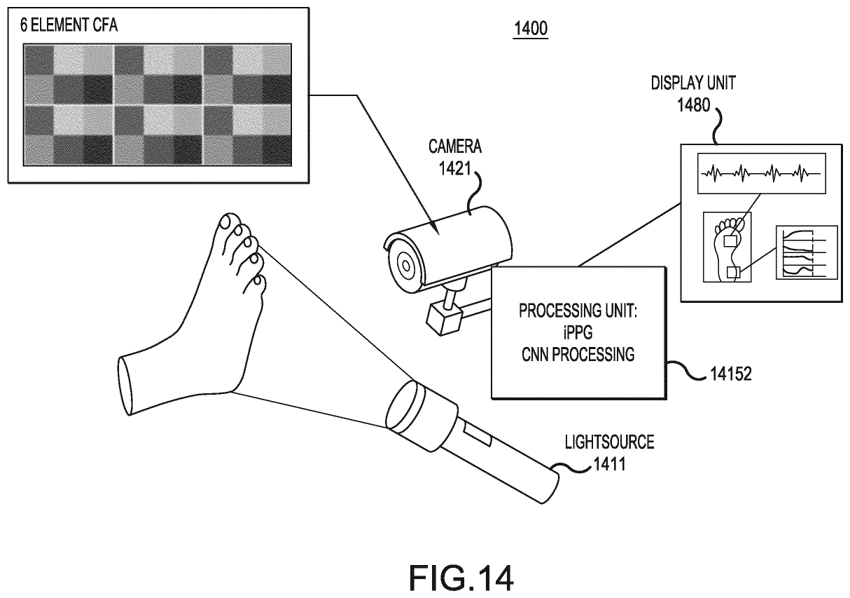

FIG. 14 illustrates another system for simultaneous measurement of tissue perfusion and tissue oxygenation, in accordance with a representative embodiment.

In FIG. 14, the system 1400 includes a light source 1411, a camera 1421, a processing unit 14152, and a display 1480. The camera 1421 incorporates a color filter array (CFA) specifically optimized for the purpose of the detection of tissue oxygenation (StO2), HbT, and metHb. The camera 1421 may therefore detect reflected light at the optimized central wavelengths all at the same time. Embodiments based on FIG. 14 additionally incorporate a broadband light source as the light source 1411.

The processing unit 14152 processes images obtained by the camera 1421. The display 1480 is a unit/user interface for displaying relevant information to a doctor.

FIG. 15 illustrates a computer system, on which a method for simultaneous measurement of tissue perfusion and tissue oxygenation is implemented, in accordance with another representative embodiment.

Referring to FIG. 15, the computer system 1500 includes a set of software instructions that can be executed to cause the computer system 1500 to perform any of the methods or computer-based functions disclosed herein. The computer system 1500 may operate as a standalone device or may be connected, for example, using a network 1501, to other computer systems or peripheral devices. In embodiments, a computer system 1500 performs logical processing based on digital signals received via an analog-to-digital converter.

In a networked deployment, the computer system 1500 operates in the capacity of a server or as a client user computer in a server-client user network environment, or as a peer computer system in a peer-to-peer (or distributed) network environment. The computer system 1500 can also be implemented as or incorporated into various devices, such as a workstation that includes the controller 150 in FIG. 1A, a stationary computer, a mobile computer, a personal computer (PC), a laptop computer, a tablet computer, or any other machine capable of executing a set of software instructions (sequential or otherwise) that specify actions to be taken by that machine. The computer system 1500 can be incorporated as or in a device that in turn is in an integrated system that includes additional devices. In an embodiment, the computer system 1500 can be implemented using electronic devices that provide voice, video or data communication. Further, while the computer system 1500 is illustrated in the singular, the term “system” shall also be taken to include any collection of systems or sub-systems that individually or jointly execute a set, or multiple sets, of software instructions to perform one or more computer functions.

As illustrated in FIG. 15, the computer system 1500 includes a processor 1510. The processor 1510 may be considered a representative example of the processor 152 of the controller 150 in FIG. 1B and executes instructions to implement some or all aspects of methods and processes described herein. The processor 1510 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 1510 is an article of manufacture and/or a machine component. The processor 1510 is configured to execute software instructions to perform functions as described in the various embodiments herein. The processor 1510 may be a general-purpose processor or may be part of an application specific integrated circuit (ASIC). The processor 1510 may also be a microprocessor, a microcomputer, a processor chip, a controller, a microcontroller, a digital signal processor (DSP), a state machine, or a programmable logic device. The processor 1510 may also be a logical circuit, including a programmable gate array (PGA), such as a field programmable gate array (FPGA), or another type of circuit that includes discrete gate and/or transistor logic. The processor 1510 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 1500 further includes a main memory 1520 and a static memory 1530, where memories in the computer system 1500 communicate with each other and the processor 1510 via a bus 1508. Either or both of the main memory 1520 and the static memory 1530 may be considered representative examples of the memory 151 of the controller 150 in FIG. 1B, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 1520 and the static memory 1530 are articles of manufacture and/or machine components. The main memory 1520 and the static memory 1530 are computer-readable mediums from which data and executable software instructions can be read by a computer (e.g., the processor 1510). Each of the main memory 1520 and the static memory 1530 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, Blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 1500 further includes a video display unit 1550, such as a liquid crystal display (LCD), an organic light emitting diode (OLED), a flat panel display, a solid-state display, or a cathode ray tube (CRT), for example. Additionally, the computer system 1500 includes an input device 1560, such as a keyboard/virtual keyboard or touch-sensitive input screen or speech input with speech recognition, and a cursor control device 1570, such as a mouse or touch-sensitive input screen or pad. The computer system 1500 also optionally includes a disk drive unit 1580, a signal generation device 1590, such as a speaker or remote control, and/or a network interface device 1540.

In an embodiment, as depicted in FIG. 15, the disk drive unit 1580 includes a computer-readable medium 1582 in which one or more sets of software instructions 1584 (software) are embedded. The sets of software instructions 1584 are read from the computer-readable medium 1582 to be executed by the processor 1510. Further, the software instructions 1584, when executed by the processor 1510, perform one or more steps of the methods and processes as described herein. In an embodiment, the software instructions 1584 reside all or in part within the main memory 1520, the static memory 1530 and/or the processor 1510 during execution by the computer system 1500. Further, the computer-readable medium 1582 may include software instructions 1584 or receive and execute software instructions 1584 responsive to a propagated signal, so that a device connected to a network 1501 communicates voice, video or data over the network 1501. The software instructions 1584 may be transmitted or received over the network 1501 via the network interface device 1540.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, simultaneous measurement of tissue perfusion and tissue oxygenation enables use of an optimized selected set of wavelengths to ensure accurate and efficient estimations of the tissue perfusion and tissue oxygenation. The teachings herein include optimizing the smallest set of central wavelengths to use to enable simultaneous real-time measurements of tissue perfusion and tissue oxygenation. The smallest set of central wavelengths that ensure accuracy is identified, and can be implemented as preset central wavelengths for a camera to detect, or for a filter to individually or collectively isolate. The smallest set of central wavelengths ensures accuracy for tissue oxygenation, total hemoglobin and methemoglobin simultaneously (in parallel), independent from interferences from other parameters such as e.g. scattering, water and fat.

Although simultaneous measurement of tissue perfusion and tissue oxygenation has been described with reference to several exemplary embodiments, it is understood that the words that have been used are words of description and illustration, rather than words of limitation. Changes may be made within the purview of the appended claims, as presently stated and as amended, without departing from the scope and spirit of simultaneous measurement of tissue perfusion and tissue oxygenation in its aspects. Although simultaneous measurement of tissue perfusion and tissue oxygenation has been described with reference to particular means, materials and embodiments, simultaneous measurement of tissue perfusion and tissue oxygenation is not intended to be limited to the particulars disclosed; rather simultaneous measurement of tissue perfusion and tissue oxygenation extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A system, comprising:
a controller comprising a memory that stores instructions and a processor that executes the instructions;
a light source that emits emission light towards tissue at six or more predetermined central wavelengths; and
a camera that detects reflected light reflected from the tissue based on the emission light,
wherein, when executed by the processor, the instructions cause the system to:
measure tissue perfusion based on the reflected light; and
measure tissue oxygenation based on the reflected light.

2. The system of claim 1, further comprising:
a filter that simultaneously filters the reflected light to the six or more predetermined central wavelengths.

3. The system of claim 2, wherein the filter simultaneously filters the reflected light to six predetermined central wavelengths.

4. The system of claim 1, wherein the light source emits broadband emission light that includes light at the six or more predetermined central wavelengths.

5. The system of claim 1, further comprising:
a filter that filters the reflected light to the six or more predetermined central wavelengths one at a time.

6. The system of claim 5, wherein the filter filters the reflected light to six predetermined central wavelengths.

7. The system of claim 5, wherein the filter comprises a motorized filter wheel.

8. The system of claim 1,
wherein the tissue perfusion and the tissue oxygenation are measured based on the reflected light independent of interferences due to characteristics of the tissue.

9. The system of claim 1,
wherein the six or more predetermined central wavelengths are optimized for simultaneous detection of tissue perfusion, tissue oxygenation, total hemoglobin and methemoglobin, independent of interferences due to characteristics of the tissue, and
wherein, when executed by the processor, the instructions further cause the system to:
apply a trained convolutional neural network to the reflected light to obtain an output comprising an estimate of the tissue perfusion, an estimate of tissue oxygenation, an estimate of total hemoglobin and an estimate of methemoglobin.

10. The system of claim 1, wherein the light source comprises a multi-wavelength light source.

11. The system of claim 1, wherein the light source comprises a light-emitting diode (LED) light source.

12. The system of claim 1, wherein the light source comprises lasers at the predetermined central wavelengths.

13. The system of claim 1, wherein the camera is configured to perform imaging photoplethysmography (PPG).

14. The system of claim 1, wherein the camera comprises a multispectral camera configured to detect the six or more central wavelengths as a fixed configuration.

15. The system of claim 1, further comprising:
a filter,
wherein the camera comprises a multispectral camera and the filter is configured to filter the reflected light to the six or more central wavelengths as a fixed configuration.

16. The system of claim 1, wherein the camera comprises a hyperspectral camera configured to detect the six or more central wavelengths as a tunable configuration.

17. The system of claim 1, further comprising:
a filter,
wherein the camera comprises a hyperspectral camera and the filter is configured to filter the reflected light to the six or more central wavelengths as a tunable configuration.
